(19) United States
(12) Patent Application Publication
(10) Pub. No.: US 2025/0243418 A1
(43) Pub. Date: Jul. 31, 2025

(54) RENEWABLE KEROSENE FUEL HAVING EXCELLENT LOW-TEMPERATURE PROPERTIES

(71) Applicant: AXENS, Rueil-Malmaison (FR)

(72) Inventor: Pierre-Antoine GEORG, Rueil-Malmaison (FR)

(73) Assignee: AXENS, Rueil-Malmaison (FR)

(21) Appl. No.: 18/854,280

(22) PCT Filed: Apr. 4, 2023
(86) PCT No.: PCT/EP2023/058742

§ 371 Date: Oct. 4, 2024

(30) Foreign Application Priority Data
Apr. 5, 2022 (FR) FR2203113

(51) Int. Cl. C10L 1/04

(52) U.S. Cl. CPC C10L 1/04; C10L 2270/04

(57) ABSTRACT

The present invention concerns a kerosene base comprising at least 60.0 weight % of a mixture composed of C3n hydrocarbons and C4n hydrocarbons, where n is a natural integer chosen from 3 and 4, at least 80 weight % of the total weight of the kerosene base being isoparaffins, said kerosene base having excellent low-temperature properties. The present invention also concerns any composition comprising said kerosene base, a process for preparing it and its use as a fuel in the aviation sector.

### TECHNICAL FIELD

The present invention pertains to the field of biobased fuels and concerns more especially a kerosene base which preferably is renewable and meets the specifications in force, in particular those defined in the ASTM D7566 standard and more particularly in Annex 5, and very advantageously has particularly satisfactory low-temperature properties. The present invention also concerns any composition comprising such a kerosene base.

### PRIOR ART

Airline companies have committed to carbon-neutral growth particularly in commercial aviation from 2021, and American airline companies have set an objective to reduce emissions of CO2 by 50% in 2050 relative to 2005 levels. However, the improvements in aircraft and engine efficiency are not proving to be sufficient to attain carbon neutrality. Sustainable aviation fuels (or SAF) therefore appear to be critical for achieving this objective.

It seems necessary, therefore, to develop at least partly biobased kerosenes that have properties at least equivalent to those of fossil kerosenes.

Hence patent U.S. Pat. No. 8,373,012 proposes a method for preparing mixtures of renewable fuels, comprising converting fermentative isobutanol to synthesized paraffinic kerosene (SPK), which meet the specifications of the ASTM D7566-10a standard, Annex 1, and so have more particularly a freezing point of at most −40° C.

Patent application WO13085980 discloses a renewable kerosene fuel derived at least partly from biomass and comprising between 5 and 20 weight % of isoparaffins and between 15 and 95 weight % of naphthenes. More especially, document WO13085980 describes a renewable kerosene fuel derived from biomass and having a freezing point possibly of around −39° C., −40° C. or −70° C. and more particularly a density at 15° C. (i.e. 60° F.) of between 819 and 839 kg/m³ (between 0.8192 g/cc and 0.8393 g/cc). Said kerosene fuel comes from a fuel composition which is predominantly n-paraffinic (more than 40 weight %), comprising around 7 weight % of C9 compounds, 12 weight % of C10 compounds, 8 weight % of C11 compounds, 9 weight % of C12 compounds and around 11% of C14+ compounds, so corresponding to a C9+ mixture comprising around 35 weight % of C9 and C12 compounds.

Patent application WO18224730 in turn discloses a renewable kerosene fuel compound, more particularly obtained by a Fischer-Tropsch process, comprising predominantly isoparaffins and typically predominantly C15 to C18 paraffins, C15-paraffins (thus comprising fewer than 15 carbon atoms) being present in an amount of less than 20 weight %, with a distillation range more particularly between 145° C. and 280° C. and a freezing point of around −51° C. WO18224730 also discloses compositions comprising a renewable kerosene component of this kind in a mixture with a kerosene of fossil origin (i.e. from petroleum) having a freezing point of less than or equal to −40° C., more particularly ranging between around −53° C. and around −55° C.

Patent application WO2022/008534 describes renewable fuel products composed predominantly of isoparaffins (at least 86.7 weight %) and comprising between 35.4 and 69.8 weight % of C9-C12 paraffins (n- and iso-paraffins), i.e. paraffins comprising between 9 and 12 carbon atoms, in other words C9, C10, C11 and C12 paraffins. More especially, document WO2022/008534 describes a renewable kerosene component comprising 86.7 weight % of isoparaffins and composed to an extent of 69.8 weight % of C9-C12 paraffins (n- and iso-paraffins), made up of 33.5 weight % of C9 and C12 paraffins, 19.5 weight % of C10 paraffins and 16.8 weight % of C11 paraffin, having a freezing point of −54° C. and a density of 750.7 kg/m³.

However, none of the prior-art documents describes kerosenes, and more particularly kerosenes which are at least partly biobased, which meet all of the specifications in force, in particular a density of between 730 and 770 kg/m³ at 15° C. and a flash point of greater than or equal to 38° C., and more particularly which have excellent low-temperature properties, more especially having a very low freezing point and in particular less than or equal to −60° C., preferably less than or equal to −80° C.

### SUMMARY OF THE INVENTION

Hence the present invention relates to a kerosene base comprising at least 60.0% by weight of a mixture composed of C3n hydrocarbons and C4n hydrocarbons, where n is a natural integer chosen from 3 and 4, wherein the kerosene base comprises at least 80% by weight isoparaffins, relatively to the total weight of the kerosene base.

The advantage of the present invention lies in the substantial improvement in the low-temperature properties of the kerosenes, more particularly of mixtures of kerosenes for aircraft engines, meeting all of the other specification criteria for kerosenes, more particularly those intended for aviation, and more especially the specifications of the ASTM D7566 standard and in particular those of Annex 5 of the ASTM D7566 standard, such as in particular a flash point of greater than or equal to 38° C. and a density of between 730 and 770 kg/m³ at 15° C. Indeed, the kerosene base according to the present invention has a very low freezing point, in particular of less than or equal to −60° C., more especially less than or equal to −70° C., preferably less than or equal to −80° C., and the mixtures comprising it have satisfactory freezing points which meet the specification in force, with a freezing point of less than or equal to −40° C.

Another advantage of the present invention lies in the fact that the kerosene base according to the invention, used alone or as a mixture with other kerosenes of biobased and/or fossil origin, is advantageously at least partly biobased, which will assist the airline companies to attain their objective of reducing their CO2 emissions and hence their carbon footprint.

### DESCRIPTION OF THE EMBODIMENTS

According to the present invention, the expressions “of between . . . and . . . ” and “between . . . and . . . ” are equivalent and mean that the limiting values of the interval are included in the described range of values. If such is not the case and if the limiting values are not included in the range described, such information will be introduced by the present invention.

For the purposes of the present invention, the different ranges of parameters given for the various characteristics may be used alone or in combination.

In the continuation of the text, specific embodiments of the invention may be described. They can be implemented separately or combined together, without limitation of combinations when this is technically feasible.

The term “biobased” means that the product/compound which it qualifies is an organic product/compound whose carbon originates from CO2 present in the atmosphere that has been fixed recently (on the human scale) by means of solar energy (photosynthesis). Terrestrially, this CO2 is captured or fixed by plant life (for example, agricultural crops or forest materials). In the oceans, the CO2 is captured or fixed by photosynthesizing bacteria or phytoplankton. For example, a biobased material has a 14C/12C isotope ratio of more than 0. Conversely, a material of fossil origin has a 14C/12C isotope ratio of around 0. The terms “renewable” or “obtained from renewables” may also be used. To determine if a product/compound is biobased or obtained from renewables, its modern carbon content (or percent modern carbon, pMC) is measured according to the ASTM D 6866-21 standard (“Determining the biobased content of natural-range materials via analysis by isotope and radiocarbon ratio mass spectrometry”). The method of this standard indeed measures the 14C/12C isotope ratio in a sample and compares it with the ¹⁴C/¹²C isotope ratio of a standard biobased reference to give the percentage biobased content of the sample, the reference giving a radiocarbon content roughly equivalent to the fraction of atmospheric radiocarbon in 1950. The pMC of the standard biobased reference material therefore equals 100%. The pMC of a material of fossil origin is around 0%. The pMC of a biobased material is strictly greater than 0%, for example greater than or equal to 1%. A current biobased material may therefore also possibly have a pMC of more than 100%.

In the present description, the terms “T95” or “T95 temperature” are interchangeable and denote the temperature at which 95% by weight of the product in question has evaporated. It is determined according to the standardized method ASTM D2887. In parallel, “T5” or “T5 temperature” is the temperature at which 5% by weight of the product in question has evaporated, determined according to the same standardized method ASTM D2887.

In the present description, the term “Cx” denotes compounds including x carbon atoms. For example, a C3 chemical compound contains 3 carbon atoms. The term “Cx+” denotes compounds having at least x carbon atoms. For example, C9+ compounds are compounds containing at least 9 carbon atoms (i.e. 9 or more carbon atoms). The term “Cx−” denotes compounds having at most x carbon atoms.

In the present description, the terms “3n carbon atoms” or “C3n” and “4n carbon atoms” or “C4n”, where n is a natural integer chosen from 3 or 4, mean respectively 3×n (literally three multiplied by n) and 4×n (literally four multiplied by n) carbon atoms, i.e.:
when n=3, 3×3=9 and 4×3=12 carbon atoms (i.e. C9 and C12),
when n=4, 3×4=12 and 4×4=16 carbon atoms (i.e. C12 and C16).

Similarly, the terms “5n carbon atoms” or “C5n”, where n is a natural integer chosen from 3 or 4, mean 5×n (literally five multiplied by n) carbon atoms, i.e.: when n=3, 5×3=15 carbon atoms (i.e. C15), and when n=4, 5×4=20 (i.e. C20).

According to the present invention, the terms “olefin” and “monoolefin” are used without distinction from one another and refer to hydrocarbons comprising a single double bond.

In the present description, the smoke point is a parameter determined by a standardized test described in the ASTM D1322/IP 598 standard, which involves measuring the maximum height of a flame emitting no smoke in an oil lamp (lamp with wick). The smoke point is expressed in mm. A higher smoke point indicates a low C/H ratio, wherein C/H ratio means the ratio between carbon C atoms and hydrogen H atoms, and correlates to better kerosene qualities, more particularly a product of greater thermal stability. The smoke point is the temperature from which the oils or fats emit smoke continuously. Beyond this temperature, the products begin to decompose and become denatured. For kerosene, a low C/H ratio (or a high H/C ratio) is preferred since kerosene must have high specific energy. Moreover, high C/H ratio implies higher flame radiation, increase of carbon deposit in aircraft engines and thus increase of the smoke point.

The freezing point of a substance defines a temperature at which the liquid and solid states of the substance are able to coexist in equilibrium (ASTM D5972 and/or D7153).

More especially, the present invention relates to a kerosene base comprising, preferably consisting of:
at least 60.0% by weight, preferably at least 70.0% by weight, possibly at least 75.0% by weight, more preferably at least 80.0% by weight, or even at least 90.0% by weight (100% by weight being the maximum), of a mixture composed of, preferably consisting of, hydrocarbons containing 3n carbon atoms (C3n hydrocarbons) and hydrocarbons containing 4n carbon atoms (C4n hydrocarbons), n being a natural integer chosen from 3 and 4, the mixture being therefore composed advantageously of hydrocarbons containing 9 and 12 carbon atoms (i.e. C9 and C12 hydrocarbons) or hydrocarbons containing 12 and 16 carbon atoms (i.e. C12 and C16 hydrocarbons),
at least 80% by weight, preferably at least 90% by weight, very preferably at least 95% by weight, of the total weight of the kerosene base being isoparaffins.

Advantageously, the kerosene base comprises predominantly aliphatic, i.e. predominantly non-cyclic and non-aromatic, hydrocarbons; preferably the kerosene base comprises at least 90% by weight, preferably at least 95% by weight, more preferably at least 99% by weight of aliphatic hydrocarbons. Preferably, the kerosene base comprises less than 10% by weight, preferably less than 5% by weight, more preferably less than 1.0% by weight and very preferably less than 0.5% by weight of cyclic and/or aromatic hydrocarbon compounds, such as naphthene, benzene and/or naphthalene compounds. In a preferred manner, the kerosene base comprises strictly less than 10% by weight naphthene compounds (which are also called cyclo-paraffines), preferably less than 5% by weight, more preferably less than 1.0% by weight, preferentially less than 0.5% by weight, and in a very preferred manner is free of naphthene compounds. Indeed, naphthene compounds increase density of the produced kerosene and have a C/H ratio between carbon atoms and hydrogen atoms greater than the one of paraffines.

Very advantageously, the kerosene base comprises predominantly hydrogenated aliphatic hydrocarbons, called alkanes or else paraffins, meaning that the kerosene base comprises preferably at least 90% by weight, more preferably at least 95% by weight, more preferably still at least 99% by weight, of paraffins, i.e. of linear paraffins (or n-paraffins) and branched paraffins (or isoparaffins). More particularly, the C3n and C4n hydrocarbons in the mixture of the kerosene base are predominantly hydrogenated aliphatic hydrocarbons, i.e. preferably at least 90% by weight, more preferably at least 95% by weight, more preferably still at least 99% by weight, of C3n and C4n paraffins.

The kerosene base may optionally comprise olefins, more particularly C3n and C4n olefins, preferably in an amount by weight of less than 5% by weight, preferably less than 1.0% by weight, very preferably less than 0.5% by weight.

Preferably, the kerosene base comprises predominantly branched paraffins (or isoparaffins), i.e. comprises at least 80% by weight, preferably at least 90% by weight, more preferably at least 95% by weight, of branched paraffins (or isoparaffins). Preferentially, the C3n and C4n hydrocarbons in the mixture of the kerosene base according to the invention are predominantly branched hydrogenated hydrocarbons, thus being at least 80% by weight, preferably at least 90% by weight, very preferably at least 95% by weight, C3n and C4n isoparaffins, more particularly C9 and C12 isoparaffins or C12 and C16 isoparaffins. Very advantageously, the isoparaffins, or branched paraffins, are predominantly multi-branched. Preferentially, therefore, the kerosene base comprises at least 80% by weight, preferably at least 90% by weight, very preferably at least 95% by weight, of isoparaffins, and advantageously at least 40% by weight, preferably at least 50% by weight, more preferably at least 70% by weight, of multi-branched paraffins. The term “multi-branched paraffins” means that said paraffins have a branching index of greater than or equal to 2 and preferably less than or equal to 9, very preferably less than or equal to 6. Very preferably, the kerosene base comprises at least 60.0% by weight, preferably at least 70.0% by weight, more preferably at least 80.0% by weight, more preferably still at least 90.0% by weight, of a mixture of C3n and C4n isoparaffins, and more particularly of a mixture of C9 and C12 isoparaffins or of a mixture of C12 and C16 isoparaffins, and very advantageously at least 40% by weight, preferably at least 50% by weight, more preferably at least 70% by weight, of multi-branched C3n and C4n paraffins and more particularly of a mixture of multi-branched C9 and C12 paraffins or of a mixture of multi-branched C12 and C16 isoparaffins.

Very preferably, the kerosene base comprises at most 10% by weight of n-paraffins, preferably at most 7% by weight of n-paraffins and more preferably at most 5% by weight of n-paraffins, and may for example comprise at least 2% by weight of n-paraffins.

The hydrocarbons containing 3n and 4n carbon atoms are preferably present in the kerosene base in amounts by weight such that the weight ratio C4n/C3n of the C4n hydrocarbons to the C3n hydrocarbons is greater than or equal to 0.10 and preferably less than or equal to 1.1, preferably less than or equal to 0.9, more preferably less than or equal to 0.5.

Advantageously, the kerosene base comprises preferably less than 40% by weight, preferably less than 30% by weight, possibly less than 25% by weight, more preferably less than 20% by weight, or even less than 10% by weight, of hydrocarbons containing m carbon atoms, m being a natural integer different from the integers 3n and 4n, n being as defined above, i.e. a natural integer chosen from 3 and 4, meaning that m is other than 9 and 12 or than 12 and 16. In other words, the kerosene base comprises preferably less than 40% by weight, preferably less than 30% by weight, possibly less than 25% by weight, more preferably less than 20% by weight, or even less than 10% by weight, of Cm hydrocarbons, Cm being other than C9 and C12 or than C12 and C16. In particular, the kerosene base comprises preferably less than 40% by weight, preferably less than 30% by weight, possibly less than 25% by weight, more preferably less than 20% by weight, or even less than 10% by weight, of C8−, C10, C11 and C13+ or of C11−, C13, C14, C15 and C17+ hydrocarbons, respectively when the mixture comprises, preferably consists of, C9, C12 or C12, C16 hydrocarbons. The kerosene base may optionally comprise C5n hydrocarbons, n being as defined above, thus corresponding to C15 or C20 hydrocarbons, preferably in an amount of less than or equal to 15% by weight, preferably less than or equal to 10% by weight, preferably less than or equal to 5% by weight.

Advantageously, the kerosene base according to the invention has an initial boiling temperature of greater than or equal to 140° C.

The kerosene base is advantageously at least partly biobased, preferably entirely. Preferably, the kerosene base according to the invention has a percentage of modern carbon (pMC) of greater than or equal to 1%, preferably greater than or equal to 50%, more preferably greater than or equal to 75%, more particularly greater than or equal to 90%, or even of greater than or equal to 100%.

The kerosene base described above is preferably obtained by a process comprising a step of oligomerizing C3 (containing 3 carbon atoms) to C6 (containing 6 carbon atoms), preferably C3 and/or C4, olefins in the presence of a preferably heterogeneous oligomerization catalyst, and a step of hydrogenating at least part of the reaction effluent from the oligomerization step. Said olefins come advantageously from a process for dehydrating alcohols, more particularly C3 to C6 and preferably C3 and/or C4 alcohols, said alcohols being preferably biobased, produced for example by fermentation of sugars. The skilled person knows how to adjust the conditions of temperature, pressure and feed flow rates, depending in particular on the olefinic feed and the nature of the oligomerization catalyst used. For example, the oligomerization step may be performed in the presence of silica-alumina, used as an oligomerization catalyst, at a temperature of between 20° C. and 300° C., preferably between 25 and 220° C., more preferably between 30° C. and 200° C., a pressure of between 1.5 and 6.5 MPa, preferably between 2.0 and 4.0 MPa, and an HSV (hourly space velocity, corresponding to the volume flow rate of the olefinic feed relative to the volume of catalyst in operation) of between 0.1 and 0.5 h⁻¹, preferably between 0.2 and 0.3 h⁻¹. The reaction effluent obtained at the end of the oligomerization step is preferably fractionated into at least one first fraction comprising dimers and trimers and a second fraction advantageously having a T5 greater than or equal to 140° C., said first fraction being advantageously at least partly recycled to the entry of the oligomerization step and the second fraction being advantageously at least partly sent to the hydrogenation step. The skilled person also knows how to adjust the operating conditions of the hydrogenation step, for example to a temperature of between 5° and 300° C., preferably between 6° and 200° C., a pressure of between 0.5 and 5.0 MPa, preferably between 1.0 and 5.0 MPa, and preferably in the presence of hydrogen preferably in an amount of between 0.5 and 3 weight % relative to the weight of the part of the second fraction that supplies the hydrogenation step.

More especially, the kerosene base described above may be obtained by a preparation process comprising, preferably consisting of:
a′) optionally a step of pretreating a C3 to C6, preferably C3 and/or C4, olefinic feed, preferably employing at least one adsorption section and/or water washing section and/or hydrotreating section and/or selective hydrogenation section;
a″) optionally a step of separating the olefinic feed to separate at least partially the C5 and C6 compounds present in said olefinic feed;
a) an oligomerization step supplied at least by the olefinic feed, optionally pretreated and/or separated, a first recycle and a second recycle, the first recycle being preferably in a weight ratio of between 0.3 and 1.5, preferably between 0.5 and 1.2, relative to the olefinic feed, and the second recycle being preferably in a weight ratio of between 0.5 and 10.0, preferably between 1.0 and 5.0 and more preferably between 1.0 and 4.0, relative to the olefinic feed, said step a) being operated in the presence of at least one preferably solid oligomerization catalyst, more particularly in the presence of silica-alumina, at a temperature of preferably between 2° and 500° C., more particularly between 20° C. and 300° C., more especially between 25 and 220° C., or even between 30° C. and 200° C., at a pressure of preferably between 1.0 and 10 MPa, more particularly between 1.5 and 6.5 MPa, more especially between 2.0 and 4.0 MPa, and an HSV of preferably between 0.1 and 0.5 h 1, more particularly between 1.5 and 6.5 MPa, more especially between 0.2 and 0.3 h 1, to produce a reaction effluent comprising dimers, trimers and oligomers;
b) a step of fractionating the reaction effluent obtained at the end of step a), into at least:
   a light fraction comprising at least part of the olefinic feed not converted in step a);
   an intermediate fraction comprising at least part of the dimers and trimers produced in step a); and
   a heavy fraction, comprising the oligomers present in the reaction effluent;
c) a recycle step, comprising: the preparation of a first recycle comprising, preferably consisting of, at least part of the light fraction from step b); the preparation of a second recycle comprising, preferably consisting of, at least part of the intermediate fraction from step b); and the transfer of the first recycle and the second recycle to the oligomerization step a);
d) a step of hydrogenating at least part of the heavy fraction separated in step b) in the presence of hydrogen, to give a hydrogenated heavy fraction comprising at least one kerosene base;
e) optionally a step of separating the hydrogenated heavy fraction, to separate at least said kerosene base according to the invention.

Very advantageously, the kerosene base according to the invention, as defined and more particularly at least partly biobased, meets the specifications in force for kerosenes, more particularly for aviation, and more especially the specifications of the ASTM D7566 standard and in particular those defined in Annex 5 of the ASTM D7566 standard. More particularly, the kerosene base has a final boiling temperature of less than or equal to 300° C. and advantageously a temperature differential T90-T10 (differential between the boiling temperature at which 10% of the product tested can be recovered and the boiling temperature at which 90% of the product tested can be recovered) of greater than or equal to 21° C., preferably greater than or equal to 40° C. Moreover, the kerosene base according to the invention has a flash point of advantageously greater than or equal to 38° C. and a density at 15° C. of preferably between 730 and 770 kg/m³. Furthermore, the kerosene base according to the invention has a freezing point of less than or equal to −40° C., more particularly less than or equal to −50° C., more especially less than or equal to −60° C., more especially still less than or equal to −70° C., or even of less than or equal to −80° C.

The present invention also relates to any composition comprising the kerosene base described above, preferably a composition comprising at least 5% by weight of said kerosene base, preferably at least 10% by weight of the kerosene base, more preferably at least 30% by weight of the kerosene base, very preferably at least 50% by weight of the kerosene base, and possibly preferably less than 90% by weight, more preferably less than 60% by weight of the kerosene base. Besides the kerosene base, said composition comprises one or more biobased kerosene products other than the kerosene base according to the invention, and/or one or more kerosene products of fossil origin (also called fossil kerosene products or non-renewable kerosene products), for example so-called aromatic kerosene products.

The present invention also relates to a process for preparing such a composition, comprising mixing the kerosene base according to the invention with at least one kerosene product other than said kerosene base, more particularly with a biobased and/or fossil kerosene product, preferably in a proportion of the kerosene base of at least 5% by weight, preferably at least 10% by weight, more preferably at least 30% by weight, very preferably at least 50% by weight, relative to the total weight of the composition. Advantageously, said process for preparing the composition also comprises all of the steps for preparing the kerosene base according to the invention as described above, prior to the mixing of said kerosene base with said at least one kerosene product other than said kerosene base.

Such compositions and the processes for preparing them have the advantage of being able to improve, advantageously simply, the low-temperature properties of the kerosene fuels, in particular those intended for aviation applications, while maintaining the other characteristics and properties of the kerosenes in the specifications in force. A further advantage of these compositions is that they have a percentage of modern carbon (pMC) of greater than or equal to 1%, preferably greater than or equal to 10%, more preferably greater than or equal to 25%, more particularly greater than or equal to 50%. Hence the compositions prepared, comprising the kerosene base according to the invention, may assist the airlines companies to attain the objectives of reducing fixed CO2 emissions, more particularly a reduction in emissions of CO2 by 50% in 2050 relative to 2005 levels, and so to attain carbon neutrality.

The present invention also relates to the use of a composition as described above as fuel for aircraft engines.

The examples that follow illustrate the invention, more particularly particular embodiments of the invention, without limiting the scope thereof.

### EXAMPLES

### Example 1 (in Accordance with the Invention)

A biobased olefinic feed comprising 94.5% by weight of isobutene and 5.5% by weight of isobutane is oligomerized in the presence of a silica-alumina catalyst, at a temperature between 3° and 90° C., a pressure of 3.5 MPa and an HSV of 0.3 h⁻¹. The oligomerization reaction is performed in three reactors in series, with an intermediate exchanger between each reactor. A portion of the hydrogenated finished product obtained after hydrogenation is recycled to the oligomerization step, so as to control the exotherm in the reactors.

The reaction effluent obtained at the end of the oligomerization step is separated by distillation into:
a C4-cut, comprising the unreacted feed and corresponding to around 7.1% by weight of the reaction effluent, said C4-cut being returned in its entirety to the entry of the oligomerization step;
a C5-140° C. cut, corresponding to around 31.4% by weight of the reaction effluent, which is recycled in its entirety to the entry of the oligomerization step, the weight ratio of the C5-140° C. cut to the fresh biobased olefinic feed being 2.0; and
a 140-300° C. cut, corresponding to around 61.5% by weight of the reaction effluent, which is sent to a hydrogenation step.

The hydrogenation is carried out in the presence of a nickel catalyst on an alumina support, at 180° C. under 3.0 MPa of hydrogen with an HSV of 0.5 h⁻¹ and a hydrogen flow rate of 50 NL/h.

The olefin content observed after hydrogenation is very low (bromine number <0.8 g/100 g), meaning that the degree of hydrogenation is high.

The hydrogenation effluent then obtained is next sent to a distillation section. At the end of this distillation section, a kerosene cut whose distillation range is 140° C.-300° C. is obtained: it corresponds to a kerosene base. The kerosene base obtained is analysed: its characteristics and properties are presented in Table 1.

TABLE 1

| | |
|---|---|
| Amounts (in weight %) of the following compounds: | |
| C11− | 0.9 |
| C12 | 85.7 |
| C13 | — |
| C14 | — |
| C15 | — |
| C16 | 10.7 |
| C17+ | 2.7 |
| Ratio C16/C12 (weight/weight) | 0.12 |
| Amount of olefins (in weight %) | <1% |
| Amounts (in weight %) of the C12 and C16 | 96.4 |
| isoparaffins | |
| pMC | 100% |
| Distillation: | |
| Initial boiling temperature (° C.) | 168 |
| Final boiling temperature (° C.) | 282 |
| T90-T10 (° C.) | 42 |
| Density at 15° C. (kg/m³) | 759 |
| Flash point (° C.) | 42.5 |
| Freezing point (° C.) | −80° C. |

1. A kerosene base comprising at least 60.0% by weight of a mixture composed of C3n hydrocarbons and C4n hydrocarbons, where n is a natural integer chosen from 3 and 4, wherein the kerosene base comprises at least 80% by weight isoparaffins, relatively to the total weight of the kerosene base.

2. The kerosene base according to claim 1, wherein the kerosene base comprises at least 90% by weight of isoparaffins.

3. The kerosene base according to claim 1, wherein the kerosene base comprises at least 40% by weight of multi-branched paraffins.

4. The kerosene base according to claim 1, wherein the kerosene base comprises at least 70.0% by weight of a mixture of C3n and C4n isoparaffins.

5. The kerosene base according to claim 4, wherein the kerosene base comprises at least 40% by weight; of multi-branched C3n and C4n paraffins.

6. The kerosene base according to claim 1, wherein said kerosene base has a weight ratio C4n/C3n of the C4n hydrocarbons to the C3n hydrocarbons of greater than or equal to 0.10.

7. The kerosene base according to claim 1, wherein said kerosene base has an initial boiling temperature of greater than or equal to 140° C.

8. The kerosene base according to claim 1, wherein said kerosene base has having a percentage of modern carbon (pMC) of greater than or equal to 1%.

9. The kerosene base according to claim 1, wherein said kerosene base has a freezing point of less than or equal to −50° C.

10. The kerosene base according to claim 1, wherein said kerosene base is obtained by a process comprising oligomerization of C3 to C6 olefins in the presence of an oligomerization catalyst, and hydrogenation of at least part of a reaction effluent from the oligomerization.

11. A composition comprising at least 5% by weight of a kerosene base according to claim 1.

12. The composition according to the preceding claim 11, further comprising at least one biobased kerosene product other than the kerosene base and/or at least one fossil kerosene product.

13. A process for preparing a composition according to claim 11, comprising mixing the kerosene with at least one kerosene product other than said kerosene base.

14. A fuel for aircraft engines comprising a composition according to claim 11.

15. The kerosene base according to claim 1, wherein the kerosene base comprises at least 95% by weight, of isoparaffins.

16. The kerosene base according to claim 1, wherein the kerosene base comprises at least 50% by weight of multi-branched paraffins.

17. The kerosene base according to claim 1, wherein the kerosene base comprises at least 80.0% by weight of a mixture of C3n and C4n isoparaffins.

18. The kerosene base according to claim 4, wherein the kerosene base comprises at least 50% by weight of multi-branched C3n and C4n paraffins.

19. The kerosene base according to claim 1, wherein said kerosene base has a weight ratio C4n/C3n of the C4n hydrocarbons to the C3n hydrocarbons of greater than or equal to 0.10 and less than or equal to 1.1.

20. The kerosene base according to claim 1, wherein said kerosene base has a percentage of modern carbon (pMC) of greater than or equal to 50%.
